(19) United States
(12) Patent Application Publication
(10) Pub. No.: US 2012/0088997 A1
(43) Pub. Date: Apr. 12, 2012

(54) Implantable biochip for managing trauma--induced hemorrhage

(76) Inventor: Anthony Guiseppi-Elie, (US)

(21) Appl. No.: 13/317,236

(22) Filed: Oct. 12, 2011

Related U.S. Application Data
(60) Provisional application No. 61/404,904, filed on Oct. 12, 2010.

(51) Int. Cl. A61B 5/145; C12M 1/34; G01N 33/48

(52) U.S. Cl. 600/364; 422/68.1; 435/287.1; 435/287.2; 600/309; 600/365

(57) ABSTRACT

A biocompatible biosensor and transmitter device for temporary implantation prior to, during and following trauma-induced hemorrhaging detects the presence and level of at least one analyte and transmits detected data to a second, external device. Thus, a method for managing post-trauma patient outcomes includes providing such a biosensor and transmitter device, temporarily implanting the biocompatible biosensor and transmitter device intramuscularly in a trauma victim; and monitoring the presence and level of the at least one analyte detected by the biocompatible biosensor and transmitter device and transmitted to the external data receiving means.

The present application claims the benefit of prior application U.S. Ser. No. 61/404,904, filed Oct. 12, 2010.

### STATEMENT REGARDING FEDERALLY SPONSORED RESEARCH OR DEVELOPMENT

The present invention was developed with the support of funds provided by the U.S. Army Medical Research and Materiel Command's contract number DAMD 17-03-1-0172 with Clemson University.

### BACKGROUND OF THE INVENTION

Trauma induced hemorrhage that eventuates hemorrhagic shock can lead to multiple organ dysfunction syndrome (MODS) and/or eventual death. Indeed, trauma is the most likely cause of demise for individuals under 50 years of age and is implicated in 68% of battlefield fatalities. During hemorrhage excessive blood loss limits the transfer of vital nutrients and oxygen throughout the body. Hemorrhagic shock is often difficult to clearly ascertain and may be induced by physically inflicted traumatic wounds, spontaneous internal bleeding, surgeries, and childbirth. Excessive hemorrhage is generally accompanied by peripheral vasoconstriction and results in poor peripheral perfusion, increased oxygen debt, increased tissue acidosis, elevated levels of stress cytokines, and eventual multiple organ dysfunction syndrome. There is a window of time, “the golden hour”, ranging from a few minutes to several hours during which resuscitation and stabilization efforts must be brought to bear if they are to be effective. Resuscitation seeks, as an end point, to satisfy the tissue oxygen debt, eliminate tissue acidosis, clear all molecular biomarkers of physiologic stress and return aerobic metabolism in all tissues.

During hemorrhage induced trauma and following surgery, hemodynamics and physiology are quite delicate and can change rapidly. There is a need to initiate immediate and continuous monitoring of molecular indicators of physiologic stress and to report these in a timely manner such that they can make a difference to resuscitation approaches and hence also to survival outcomes. Currently, the principal approach is to measure global indicators of health, vital signs. Among these are core body temperature, mean arterial blood pressure, venous oxygenation, pH, stat systematic lactate determined from drawn blood—often from an indwelling catheter placed within a major blood vessel or in the case of reconstructive surgery of the heart, directly into the heart and exiting through the heart wall and body wall. While these gross vital signs are critically important in the evaluation and management of patient wellness, they are nonetheless subject to misinterpretation. Recent work has confirmed that pre-hospital patient assessments that rely on traditional vital signs may frequently underestimate elevated lactate levels. The clinical implications suggest an emphasis on outcomes analyses. However, such outcomes must be better associated with bioanalytical measures of appropriate biomarkers that serve as prognostic indicators of MODS.

Lactic acid exists in equilibrium with glucose and accumulates within the tissues under conditions of hypoxia. Evidence has been compiled supporting the hypothesis that metabolites such as lactate and glucose experience widely altered levels as a result of hemorrhage. Lactic acid levels in the body have been correlated with severity of hemorrhage. Patients receiving traumatic head injuries tend to have increased levels of lactate within the central nervous system which has been considered a marker for a poor clinical outcome. It has been noted that the traumatic brain injury may be the cause of elevated lactate levels. Hypoxia damaged brains, upon reoxygenation, will begin to use systemic lactate as an energy source to recover synaptic function, thus subsequently, it has been considered to be potentially useful for treatment in a clinical setting.

By this evidence it is clear that immediate and continuous measurement of lactate and glucose may serve as a “gauge” for identifying shock states. Specifically, arterial lactate values of more than 5 mmol/l on admission to ICU have been associated with a mortality rate exceeding 80% at 30 days.

### SUMMARY OF THE INVENTION

In accordance with the present invention a multiple analyte measuring device for managing post-trauma patient outcomes is provided which includes an insulating substrate that supports a pattern of conductors, the conductors functioning as electrodes and upon which is further supported a pattern of an insulating layer, the insulating layer functioning to passivate a region of the patterned conductor and upon which pattern of electrodes and passivating insulating layer is further supported a pattern of a polymeric biorecognition layer possessing a molecular entity of biological origin that is specific to the analytes. Preferably, the substrate is selected from the class of dielectric materials including polished borosilicate glass, oxidized silicon, alumna, polyamide and is preferably polished borosilicate glass that may be 10 microns to 5,000 microns thick and is preferably 500 microns thick. Preferably, the conductors are selected from the class of electrical conductors including gold, platinum, palladium, iridium, indium tin oxide, polyaniline, polypyrrole and polythiophene and is preferably platinum that may be 1 nm to 500 nanometers thick and is preferably 100 nanometers thick. Preferably, the insulating layer that serves to passivate certain parts of the conductor electrodes is selected from the class of dielectric materials including silicon nitride, silicon oxide, spin-on-glass and is preferably silicon nitride that may be 0.1 microns to 5 microns thick and is preferably 0.5 microns thick. Generally, the pattern is independently and separately measured from an arrangement of at least two electrodes. Preferably, the electrodes comprise a working electrode, a counter electrode and a reference electrode, the working electrode being formed as a microdisc array with openings in the insulating layer that exposes the conductor beneath wherein the number density per unit area and the size of such openings may be varied. It is also preferred that the polymeric material comprises a hydrated hydrogel. Generally, the biorecognition layer of the device is synthesized to immobilize a molecular entity of biological origin being taken from the class of biorecognition molecules including DNA, RNA, enzymes, antibodies, antibody fragments, and antibody-linked enzymes, such biorecognition molecule being chemically modified to affix it within the biorecognition layer, the biorecognition molecule being affixed through covalent attachment to the polymeric network of the biorecognition layer. Preferably, the biorecognition molecule comprises an enzyme, the enzyme being chemically modified by the covalent attachment of methacryloyl groups. Most preferably, the enzyme is selected from lactate oxidase and glucose oxidase.

The present invention is also directed to a method for the fabrication of such a device wherein the device is cleaned, chemically modified, functionalized, the monomer of the biorecognition layer is brought into intimate contact with the pattern of electrodes of the device and the fixing and formation of the biorecognition layer by chemical reaction means. Preferably, the method for the generation of signals from the device includes the application of a voltage to the electrodes sufficient to generate a current measurable by a current measuring instrument.

In a further embodiment the present invention is directed to a biocompatible biosensor and transmitter device for temporary implantation prior to, during and following trauma-induced hemorrhaging, the device detecting the presence and level of at least one analyte and transmitting detected data to a second, external device. In one preferred embodiment, the at least one analyte is lactic acid. In another preferred embodiment the at least one analyte is glucose. In yet another preferred embodiment the at least one analyte is oxygen. In yet another preferred embodiment the at least one analyte comprises H⁺ cations for detecting pH.

The present invention is also directed to a method for managing post-trauma patient outcomes which includes the steps of: providing a biocompatible biosensor and transmitter device and a data receiving means, the biocompatible biosensor and transmitter device capable of detecting the presence and level of at least one analyte and transmitting detected data to the data receiving means; temporarily implanting the biocompatible biosensor and transmitter device intramuscularly in a trauma victim; and monitoring the presence and level of the at least one analyte detected by the biocompatible biosensor and transmitter device and transmitted to the data receiving means.

More specifically the present invention is directed to an analyte measuring device for monitoring physiological status by measuring at least one analyte, the device including an insulating substrate, a conductive material supported on the insulating substrate, an insulating layer overlying the conductive material, the insulating layer formed in a pattern thereby passivating covered surface portions of the underlying conductive material and exposing predetermined surface portions of the conductive material, the exposed portions of the conductive material functioning as electrodes, a polymeric biorecognition layer immediately adjacent to the insulating layer and the exposed surface portions of the conductive material, the polymeric biorecognition layer containing at least one molecular entity of biological or biomimetic origin that is specific to the at least one analyte being measured. The device is most preferably employed for monitoring post-trauma status. Preferably the insulating substrate is selected from dielectric materials such as polished borosilicate glass, oxidized silicon, alumina, polyamide, and silicone. Preferably the conductor is selected from gold, platinum, palladium, iridium, indium tin oxide, polyaniline, polypyrrole and polythiophene. Preferably the insulating layer is selected from dielectric materials such as silicon nitride, silicon oxide, spin-on-glass, and polyimide. It is also preferred that the polymeric recognition layer is formed of a hydrated hydrogel. Further, it is preferred the molecular entity of biological or biomimetic origin is selected from DNA, RNA, enzymes, antibodies, antibody fragments, and antibody-linked enzymes. It is also preferred that the molecular entity of biological or biomimetic origin is covalently attached to the polymer of the polymeric biorecognition layer. If the molecular entity of biological or biomimetic origin is an enzyme, it is preferred that the enzyme is chemically modified by the covalent attachment of methacryloyl groups. In such case it is preferred that the enzyme is selected from lactate oxidase, glucose oxidase, and laccase.

In another aspect the present invention is directed to a biocompatible biosensor and transmitter device for temporary implantation prior to, during and following trauma-induced hemorrhaging, the device detecting the presence and level of at least one analyte and transmitting detected data to a second, external data receiving device. Analytes which may be detected in accordance with the present invention include lactate, glucose, oxygen, and H⁺ cations for detecting pH.

The present invention is also directed to a method for managing post-trauma patient outcomes that includes the steps of providing a biocompatible biosensor and transmitter device and a data receiving means, the biocompatible biosensor and transmitter device capable of detecting the presence and level of at least one analyte and transmitting detected data to the data receiving means, temporarily implanting the biocompatible biosensor and transmitter device into a muscle of a hemorrhaging trauma victim, and employing the biosensor of the biocompatible device to collect data regarding the presence or the amount of the at least one analyte and employing the transmitter of the biocompatible device to transmit data to the data receiving means. It is preferred that the data receiving means is programmed for processing and presenting the received data.

### FIGURES OF THE DRAWING

FIG. 1A is a schematic illustration of the biochip of the biosensor and transmitter device of the present invention;

FIG. 1B illustrates the placement of the sensing sonde and biotransducer with respect to the encapsulated electronics.

FIG. 2A is an optical micrograph of the electrochemical cell-on-a-chip transducer of the present invention;

FIG. 2B is a schematic illustration of the multiple steps of surface activation, modification and derivatization for monomer casting and bioactive electroconductive hydrogel attachment in accordance with the present invention;

FIG. 3A illustrates the microdisc array electrodes before and after electropolymerization of pyrrole;

FIG. 3B is a schematic illustration of conferred biospecificity by immobilization of GOx (Channel 1, Cell A) and LOx (Channel 2, Cell B) within separate hydrogel membrane layers corresponding to Ch1 and Ch2;

FIG. 4 is a schematic illustration of the molecular constituents of a poly(HEMA-co-PEGMA-co-HMMA-co-SPMA)/P(Py-co-PyBA) electroconductive hydrogel membrane containing an oxidoreductase enzyme and illustrated with a glucose oxidase subunit along with the bioactive hydrogel topcoat of a poly(HEMA-co-PEGMA-co-MPC) containing phosphoryl choline (MPC);

FIG. 5 illustrates a trauma patient implanted with a wireless transmitting dual potentiostat to support intramuscular bioanalytical measurements of lactate and glucose in the trapezious muscle;

FIG. 6 illustrates the in vitro response of the responsive lactate and glucose biosensor of the present invention; and

FIG. 7 illustrates the in vivo amperometric response of an intramuscularly implanted lactate biosensor during hemorrhage;

### DETAILED DESCRIPTION OF THE PREFERRED EMBODIMENTS

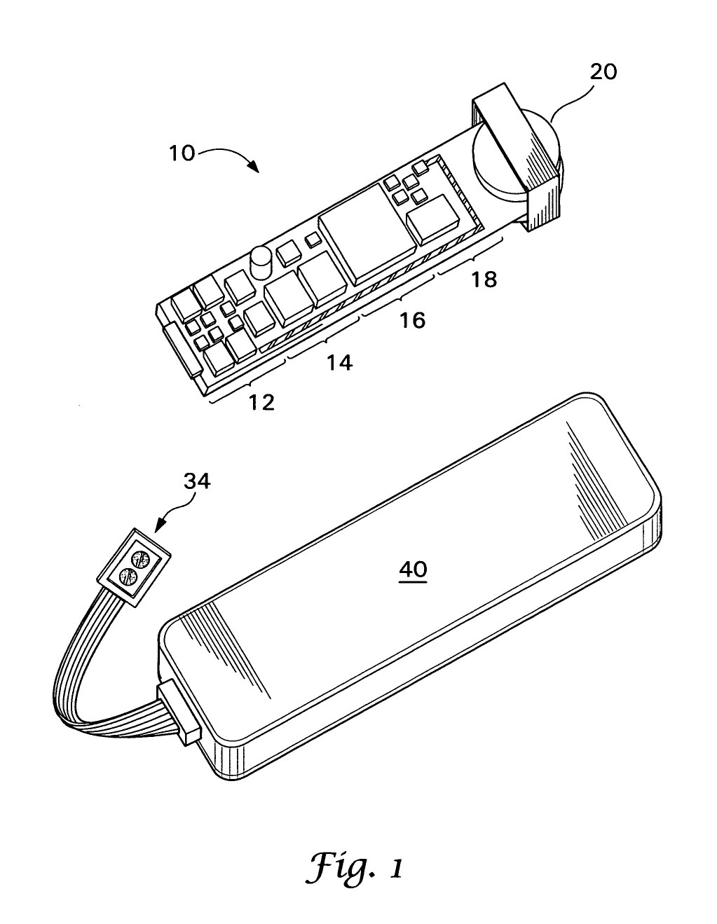

The present invention is directed to a fully integrated discrete biosensor system intended for implementation in a biochip (ASIC) format. In this sense it includes a biotransducer, associated bioinstrumentation for interrogation, capture, and processing of bioanalytical data, and a data presentation system that focuses on actionable information intended to influence patient outcomes. In its discrete design it is more appropriately described as a biosensor system. In its application specific integrated circuit (ASIC) design it is best described as a biochip. FIG. 1 shows a schematic illustration of the present biosensor system. The device 10 includes a dual potentiostat 12, data converters 14, processor 16, RF transmitter 18 and battery 20. The multiple analyte sensing sonde, that comprises a dual analyte biotransducer 34, is set distal to the encapsulated electronics 40 (containing device 10).

In a preferred embodiment the present biosensor system is capable of providing in-dwelling performance for up to six weeks. The present device has the capability to: 1) manage power usage for all system components, 2) collect and condition all analog signals from the various biotransducers of the sonde, 3) digitize those analog signals, 4) store raw or conditioned data and operational parameters, 5) wirelessly support unidirectional or bidirectional communication with a base station, and 6) be able to rapidly wake up from a low power “sleep” for immediate data collection. Specific to the needs of implantation are: 1) a very small footprint, 2) very low power consumption, and 3) wireless transmission/reception within and through living tissue.

Preferred biocompatible materials for the present system include hydrogel constituents; poly(2-hydroxyethyl methacrylate) [p(HEMA), MW=60,000: viscosity modifier], 2-hydroxyethyl methacrylate (HEMA: principal monomer), tetraethyleneglycol diacrylate (TEGDA, technical grade: crosslinker), N-[tris(hydroxymethyl)methyl]-acrylamide, (HMMA, 93%: secondary monomer), 2,2-dimethoxy-2-phenylacetophenone (DMPA, 99%: photoinitiator), pyrrole monomer (Py, reagent grade, 98+%: electroactive monomer), 4-(3′-pyrrolyl) butyric acid (PyBA:), 3-sulfopropyl methacrylate potassium salt (SPMA: anionic dopant monomer) poly(ethyleneglycol)(200)monomethacrylate (PEG200MA) poly(ethyleneglycol)(400)monomethacrylate (PEG400MA), and 2-Methacryloyloxyethyl phosphorylcholine (MPC) monomer. Prior to formulation, all of the acrylate-containing reagents were passed over an inhibitor removal column to remove the polymerization inhibitors hydroquinone and monomethyl ether hydroquinone. Tris(hydroxymethyl)aminomethane (TRIS buffer, ACS reagent, 99.8+%,) was pH-adjusted with hydrochloric acid (ACS reagent, 37%) to obtain 0.1 M buffer with pH=7.2. Ethanol (CHROMASOLV®) was used as received. Phosphate buffered saline (PBS) (0.01 M, pH7.4). All solutions were prepared with deionized (MilliQ DI) water. The diacrylate and methacrylate reagents were passed through an inhibitor removal column before use. Pyrrole was passed over an alumina silicate column removal of oligomers. The hydrogel cocktails were prepared by mixing HEMA, TEGDA, PEG(200)MA, HMMA and DMPA in a typical ratio 86:3:5:5:1 mol % to yield a base hydrogel. Acryloyl (polyethylene glycol)₁₁₀ N-hydroxy succinamide ester (acryloyl-PEG-NHS) was obtained from Nektar Therapeutics, (Huntsville, Ala.). Most other reagents used were of analytical grade and obtained from Sigma Chemical Co.

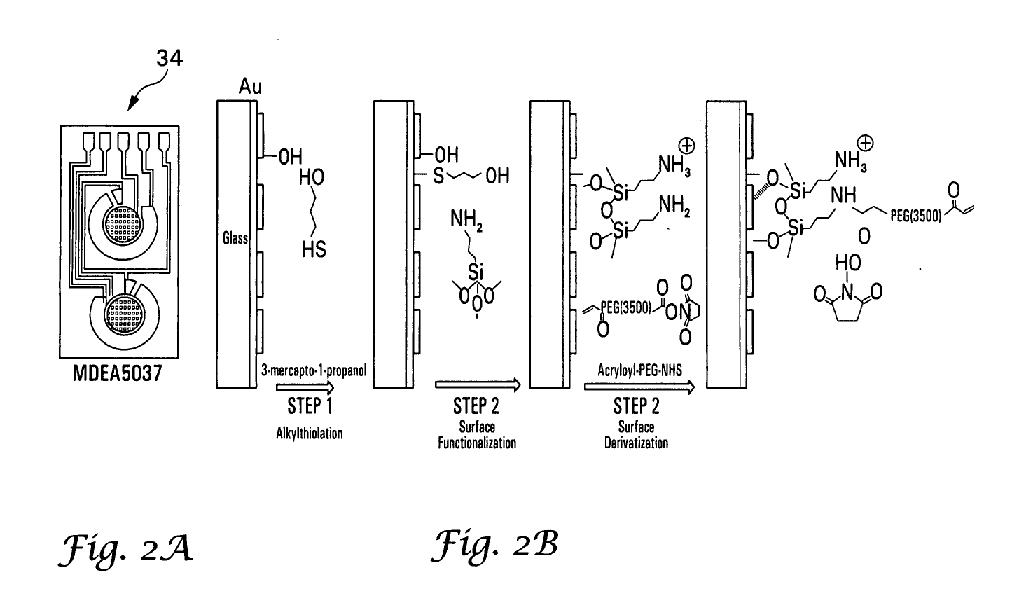

To address the need for simultaneous monitoring of interstitial glucose and lactate, a prototype dual responsive electrochemical biotransducer has been provided in accordance with the present invention. Microlithographically fabricated Electrochemical Cell-on-a-Chip Microdisc Electrode Arrays (ECC MDEA 5037-Au) were developed in conjunction with ABTECH Scientific, (Richmond, Va.). The preferred dual sensing electrochemical transducer possesses 37 recessed microdiscs arranged in a hexagonal array, each of D=50 microns diameter and total working electrode area, WEA=7.3×10⁻⁴ cm². Electrochemical transducers (0.2 cm×0.4 cm×0.05 cm) were fabricated from magnetron sputter deposited gold or e-gun vapor deposited platinum (100 nm) on an adhesion promoting titanium/tungsten (Ti/W) layer (10 nm) and on an electronics grade borosilicate glass (0.5 mm thick Schott D263). The electrodes were fashioned into two separate three-electrode electrochemical cells and these were passivated with 0.5 micron thick silicon nitride (Si₃N₄) after which the nitride layer was fluoro-plasma etched to reveal the multiple microdiscs of the working electrode, the counter electrode (7.3×10⁻³ cm²), a shared reference electrode (7.3×1e cm²) and the five bonding pads. FIG. 2A is an optical micrograph of the preferred cell-on-a-chip microdisc electrode arrays (MDEA) electrochemical transducer 34.

Electropolymerization of pyrrole and pyrrole co-polymers was achieved using a PAR 283 Galvanostat/Potentiostat in chronopotentiometric mode (galvanostatically) or chronoamperometric mode (potentiostatically) using PowerSuite software. For galvanostatic electropolymerization, current was fixed at 1 mA/cm² and defined charge densities, typically 100 mC/cm², achieved. For potentiostatic electropolymerization, voltage was fixed at 0.75 V vs. Ag/AgCl, 3MCl⁻ and defined charge densities, typically 100 mC/cm², achieved. Dynamic electrochemical characterization of the electroconductive hydrogels was studied by multiple scan rate cyclic voltammetry (MSRCV) and by electrochemical impedance spectroscopy. Both were achieved using the PAR 283 Galvanostat/Potentiostat. The latter, EIS, was achieved when the PAR 283 (AMETEK, Princeton Applied Research) was interfaced to a Solartron 1260 Frequency Response Analyzer (FRA) (AMETEK Solartron Analytical, UK). Amperometric biosensor responses to glucose and or lactate were measured at 0.70 V vs. Ag/AgCl, 3MCl⁻ in PBS 7.2 buffer at RT. Electrical characterization of the electroconductive hydrogel was done by four point conductivity measurements or by impedance spectroscopy performed on interdigitated microsensor electrodes (IME 1050.5 M-Pt—U, ABTECH Scientific, Inc. Richmond, Va.) and equivalent circuit modeling conducted within Z-View Software. Three approaches were evaluated to synthesize the electroactive polymer component within the hydrogel. In the first approach, electroactive monomer and dopant anion (SPMA) were included within the hydrogel formulation prior to membrane dip-casting. In these formulations, the Py and PyBA were typically 15M % and 1.5M % respectively and the SPMA was 5 M %. In the second approach, the transducers that were dip-coated with a base hydrogel and UV cross linked were incubated in an aqueous Py and PyBA solution and the pyrrole monomers allowed to partition into the hydrogel for at least 1 hr prior to electropolymerization. An 8:1 Py:PyBA (0.25 M:0.025 M) solution was made in DI water and its pH adjusted to 5.2 using 0.1 M Tris buffer. The third was a tandem of the forgoing two methods and this was found to be most effective in producing uniform polypyrrole within the hydrogel.

The fully assembled and packaged ECC MDEA 5037 chip was developed to allow the conduct of physiologic status monitoring studies in a small vertebrate animal (Sprague Dawley rat) hemorrhage model. To achieve this, the chip had first to be separately conferred with biospecificity to glucose (Channel 1, Cell A) and lactate (Channel 2, Cell B) through the use of molecularly engineered glucose oxidase (GOx) and lactate oxidase (LOx) to allow simultaneous measurement and monitoring of both metabolites of interest. GOx and LOx were immobilized via galvanostatic electropolymerization of pyrrole (Py) and 4-(3-pyrrolyl) butyric acid in the presence of PEGylated-GOx or PEGylated-LOx into a p(HEMA)-based hydrogel membrane layer.

To accomplish this, chips bearing gold electrodes were chemically modified using an alkane thiol (overnight in 1.0 mM 3-mercapto-1-proponal or 1.0 mM cysteamine in ethanol), the silicon nitride passivation layer subsequently modified with an organosilane (30 min immersion in 0.1 wt % 3-aminotrimethoxysilane in ethanol, rinsed in ethanol and cured at 120° C. for 20 min) and the terminal amines on both surfaces functionalized by immersion for 2 h in a solution of acryloyl(polyethyleneglycol)-N-hydroxysuccinamide (Acryloyl-PEG-NHS, MW 3500) (1.0 mM in 0.1 M HEPES at pH 8.5) that was prepared under UV free conditions. FIG. 2B schematically illustrates these surface chemical activation, modification and derivatization steps that serve to covalently attach the electroconductive hydrogel membrane layer to the transducer surface. The immobilized polyether provides multiple opportunities for concerted hydrogen bonding interaction between the surface and the hydrogel membrane. Following surface modification and derivatization, chips were then dip-coated by immersion and withdrawal from a monomer cocktail comprising HEMA, TEGDA, PEG(200)MA, HMMA, DMPA, SPMA, Py, PyBA in a typical ratio 62.5:3:5:5:2:1:5:15:1.5 mol % to yield an electroconductive hydrogel precursor. The coated transducers were immediately placed in a UV cross linker and irradiated with UV light (366 nm, 2.3 W/cm², 5 min) under an inert nitrogen atmosphere. The hydrogel membrane provides a hydrated milieu for the three dimensional bioimmobilization and hosting of the bioreceptors that confer biospecificity as well as any redox mediator that may be co-immobilized with the bioreceptor. It also serves as the reaction medium within which the electropolymerization reaction occurs.

Two approaches were evaluated to confer biospecificity to the electroconductive hydrogel membrane of this work. In the first approach the chosen oxidoreductase enzyme was included within the hydrogel formulation prior to membrane casting and so became physically entrapped as the reactive monomer became cross-linked into the hydrogel membrane and was photo-defined. In these formulations, the enzyme was typically 0.1 mg/ml. In the second approach the MDEA 5037 with its UV cross linked hydrogel membrane was incubated in an aqueous Py and PyBA that also contained the oxidase enzyme and electropolymerization was used to achieve deposition of the enzyme onto the working electrode and within the hydrogel layer. In these solutions the enzyme was typically 1 mg/ml.

GOx and LOx were separately immobilized within the supported hydrogel membrane layer on the working electrodes of the biotransducer by electropolymerization. Electropolymerization was achieved by the application of 0.70 V vs. Ag/AgCl (potentiostatic or chronoamperometric) or at 10 mA/cm² (7.3 μA galvanostatic or chronopotentiometric) to the working electrode immersed in a TRIS buffered (pH 5.2) aqueous solution containing an ad-mixture of pyrrole (0.4 M), 4-(3′-pyrrolyl)butyric acid (0.04 M) and the respective enzyme (typically 1.0 mg/ml). Then 10:1 Py:PyBA solution was made in DI water and its pH adjusted to 5.2 using 0.1 M Tris buffer. It is noteworthy that the conductive, electroactive polymer (CEP) component is allowed to initiate and grow within the immobilized hydrogel layer which serves as a multivalent macro-anion for the positively charged polypyrrole copolymer. The CEP grows from the metal|hydrogel interface and may, under poorly controlled conditions, emerge as a separate dense layer at the metal|hydrogel interface. However, the presence of the electroactive monomer and pendant dopant anions within the membrane, as well as judicious control of the reaction kinetics (current density) can result in a uniform polymer composite. ECP formation also occludes the enzyme into the gel. The enzymes, possessing a net negative charge under the electropolymerization conditions, become entrapped within the hydrogel during the electropolymerization.

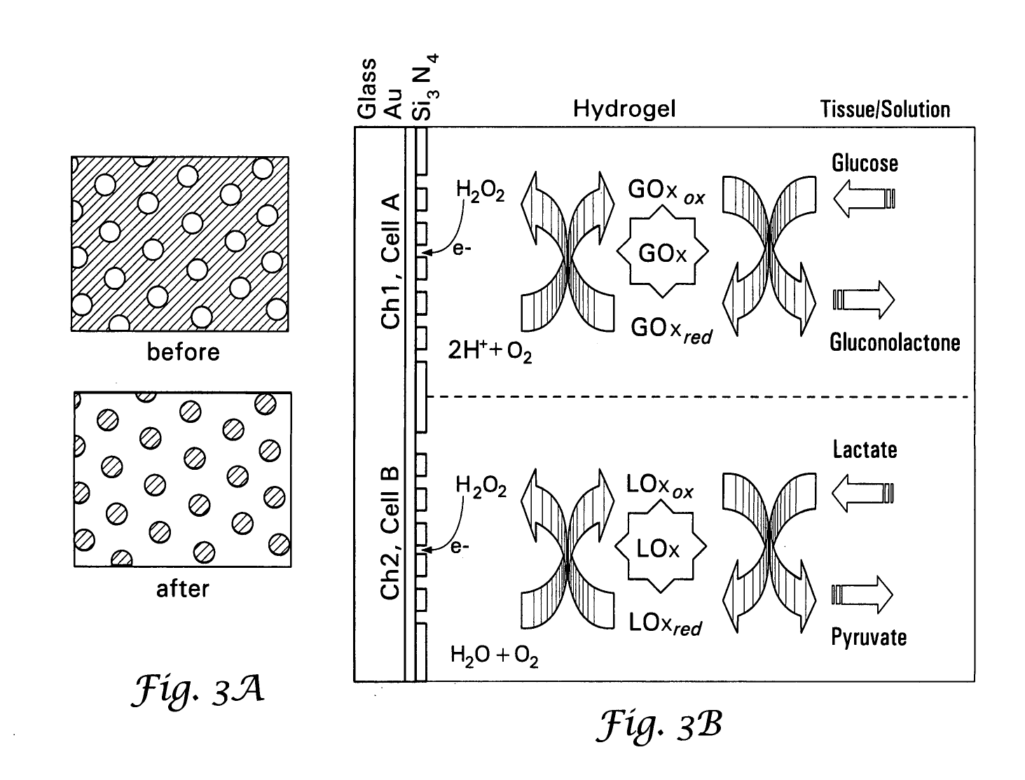

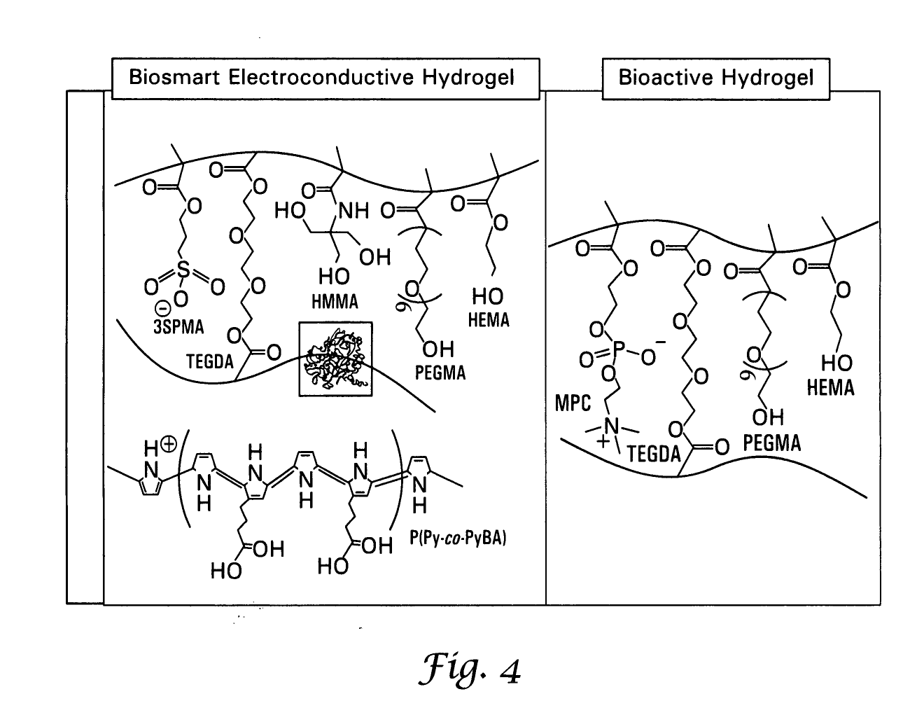

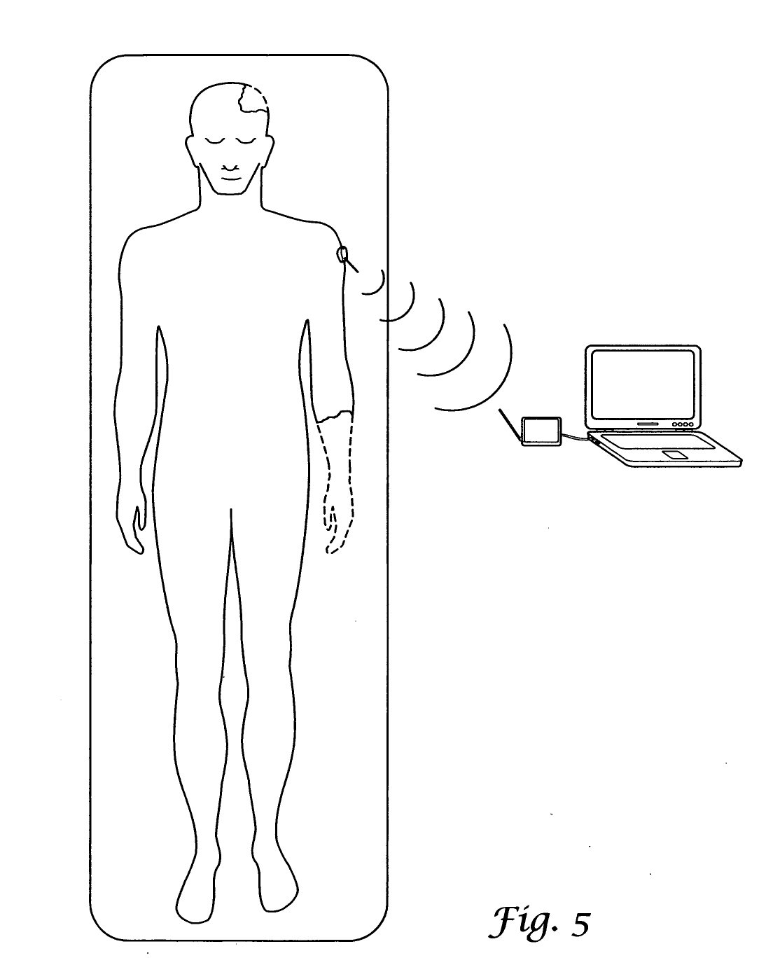

Following bioimmobilization (100 mC/cm², 100s), the chips were conditioned in TRIS buffer at 4° C. and the solution changed several times to remove un-reacted monomer. The chips were then dipped-coated again, this time into a second bioactive hydrogel cocktail formulation that contained 2-methacryloyloxyethyl phosphorylcholine (MPC or PCMA) and this UV-crosslinked to form an additional membrane layer of 3 mol % cross linked poly(HEMA-co-PEGMA-co-HMMA-co-PCMA). FIG. 3A are optical micrographs of the 50 μm diameter microdiscs of the MDEA biotransducer before and after electropolymerization of pyrrole and FIG. 3B schematically illustrates the resulting biorecognition membrane layer formed from the foregoing steps as they occur on the separate working electrodes of the MDEA biotransducer. FIG. 4 illustrates the chemistries of the biorecognition hydrogel membrane layer that subtends the transducer and the chemistries of the bioactive device-to-tissue interface hydrogel membrane layer.

In vitro amperometric calibration of the dose-response characteristics of the biotransducer was conducted at 0.7 V in 0.1 M PBKCl 7.0 at RT in response to mutarotated glucose and sodium lactate over the range 0-20 mM.

To evaluate in vitro biocompatibility of our electroconductive hydrogel outer layer, rat pheochromocytoma cells (PC12 ATCC:CRL-1721, Manassas, Va.) and human muscle fibroblasts (RMS13 ATCC:CRL-2061, Manassas, Va.) were seeded and cultured (PC12: F-12K supplemented with 2.5% fetal bovine serum (FBS) and 15% horse serum (HS), as well as 50 IU/mL penicillin and 50 μg/mL streptomycin and RMS13: RPMI supplemented with FBS (10%) and 50 IU/mL penicillin and 50 μg/mL streptomycin) on the following polymer modified planar gold electrodes: i) Au*; ii) Au*|Gel; (iii) Au*|PPy; (iv) Au*|Gel-P(Py-co-PyBA) (where A* represents the surface activated, modified and derivatized surface as in FIG. 2B) and these compared to cell growth and proliferation on a reference polystyrene cell culture surface within a 24 well plate cell cultureware. Trypsinized cells were stained with trypan blue and the final cell density determined using a hematocytometer and inverted light microscope. Cell morphology was determined following staining with rhodamine-phalloidin and DAPI subsequent to fixing with 4% paraformaldehyde. To evaluate biomaterial cytotoxicity, human aortic vascular smooth muscle cells (HA-VSMC; ATCC:CRL-1999) were cultured on PEGMA and MPC containing hydrogels that formed the outer layer of the biotransducer. HA-VSMC was cultured in F-12K (ATCC, Manassas Va.) supplemented with HEPES, TES, ascorbic acid (Sigma Aldrich, Mo.), insulin, transferrin, sodium selenite (collectively available as ITS premix, BD Bioscience), endothelial cell growth supplement (ECGS) (VWR Scientific) and supplemented with 10% fetal bovine serum (FBS) (Sigma Chemical Co).

To evaluate in vivo biocompatibility, the bioactive p(HEMA)-based hydrogels that were to form the outer device layer were tested by implantation into the trapezius muscle of Sprague Dawley rats. All animals were anaesthetized with sodium pentobarbital (35 mg/kg rat wt) administered intraperitoneally and pedal reflex used to determine the adequacy of the anesthesia. Anesthesia was then transitioned to the steroidal anesthetic Saffan (0.5 mg/kg/min) to maintain adequate level/duration of anesthesia for the individual rat and procedure. A preoperative subcutaneous dosage of atropine sulfate (100 μg/kg rat wt) was administered to decrease bronchial secretions and attenuate bradycardia following anaesthetization. Once unconscious, animals were placed in the ventral recumbent position on a heating pad with fore and hind limbs restrained with tape. After reaching a surgical plane of anesthesia, the animal's entire neck, back, hind limb and abdomen was shaved and prepared with a Betadine wash and draped with a sterile towel. The test hydrogel material was inserted in the medial trapezius muscle at the level of the mid quadriceps. The same quadracep (different area) and the opposite quadriceps area were sterilely prepped and draped to allow for placement of 1-3 additional hydrogel samples. This was done in an effort to minimize the number of animals needed to test tissue biocompatibility of the molecularly engineered hydrogels. Specimens were scored according to a histological grading scale composed of 5 categories: cell morphology, matrix staining, surface regularity, thickness of the implant material, and bonding, with a total score range from 0 to 16 (hydrogel with no foreign material accumulation being equal to 16).

Intramuscular implantation of a bioanalytical sonde must address the challenge of implant biocompatibility that arises from the eventual tissue remodeling that accompanies the trauma of implantation as well as the foreign body response. It is generally believed that the devascularized collagenous capsule that forms around the biotransducer is one important factor that compromises performance largely through its influence on the transport distances that affect substrate access. In this regard, the foregoing layered structure addresses synthesis of a soft polymeric biomaterial with low interfacial impedance, facile small molecule and ion transport, the demonstrated potential for biomolecule hosting (conferred biospecificity), and the potential for in-vivo biocompatibility. Among the multiple possible approaches to address the foreign body response are those designed to: i) emulate the chemical character of the extracellular matrix (ECM) by achieving a form of biomimicry through the use of chemical moieties such as hyaluronic acid and amino acid sequences drawn from the non-receptor binding motifs of ECM proteins (biomaterials chemistry), ii) emulate the topological character of the ECM including its chemical and physical heterogeneity (nanostructure), iii) emphasize hydrogen-bonded interactions and non-equilibrium mesostructures vs. covalently bonded isotropic materials, and iv) emulate the surface character of living cells with the use of such moieties as phosphoryl choline. The foregoing approaches pursue biomaterials design via biomimicry. The current design is an elementary approach along these lines; it seeks a highly hydrated Zwitterionic surface through the inclusion of 2-methacryloyloxyethyl phosphorylcholine to confer the biological character of the outer leaflet of cell membranes to the synthetic hydrogel. These approaches are not without detraction; among these are polymer degradation, time temperature influences and well as mechano-transduction effects.

To evaluate in vitro biocompatibility of the biomimetic hydrogel outer layer, the following physicochemical characteristics of a 3 mol % TEGDA cross-linked hydrogel that contained varying mole percentages of PEGMA and PCMA were studied. In this way, the relative contributions of pendant PEG and PC moieties to such properties as: i) hydration characteristics following 5-day equilibration was established, ii) evolving dynamic contact angles, revealing both advancing and receding contact angles, over a 5 day period, iii) adsorption of the extracellular matrix proteins; collagen, fibronectin, and laminin over a five day period to reveal protein adsorption isotherms, and iv) in vitro cell viability and proliferation using human muscle fibroblasts (RMS 13; ATCC:CRL-2061) and human aortic vascular smooth muscle cells (HA-VSMC; ATCC:CRL-1999). Hydrogels exhibited an increase in the percent hydration with an increase in the MPC content; a maximum of 93.8% increase with an increase in MPC of up to 10 mol %. PEGMA had a smaller influence on hydration than PCMA. Dynamic contact angles (θₐ=θᵣ for ideal surfaces) of as-cast hydrogel membranes were initially high (θₐ=θᵣ=ca.45°, somewhat hydrophobic) and reflected homogeneity (θₐ−θᵣ=1°). However, with increased pre-conditioning of p(HEMA) hydrogels in DI water, the dynamic contact angles showed considerable change after five days (θₐ=47°, θᵣ=22°, becoming more hydrophobic on advancing and more hydrophilic on receding) and reflected increased heterogeneity (θₐ−θᵣ=25°. However, when p(HEMA) was made to contain 10 mol % PCMA, the dynamic contact angles showed considerable decrease (θₐ=20°, θᵣ=17°, becoming more hydrophilic on both advancing and receding) and reflected increased homogeneity (θₐ−θᵣ=3°. This temporal character, while not obtained under physiologic or even physiologic-like conditions, does suggest an area for future research in the development and use of soft condensed biomaterials, that of the temporal evolution of chemical character pursuant to molecular rearrangements of the surfaces. Adsorption of FITC-dye tagged fibronectin from solutions that were 0.0 (blank), 0.1 ng and 1.0 ng/ml of protein at 25 ° C. followed the Langmuir adsorption isotherm with K_{d} and Qₘ quantitatively confirming the progressive reduction in protein adsorption when the hydrogel was pre-conditioned for varying periods (up to 5 days) in DI water and also when the MPC content was increased. There was found a strong correlation (R²=89%) between the hydration levels of the hydrogels and the ability of the hydrogel to mitigate protein adsorption and that this was manifest through the PCMA content rather than the PGMA content. Cytotoxicity studies using human aortic vascular smooth muscle cells (HA-VSMC; ATCC; CRL-1999) produced greater than 80% viability for all the hydrogel formulations. With RMS 13 cells, trypsinization and enumeration resulted in cell retention within the hydrogel matrix. This was studied by harvesting human muscle fibroblasts seeded on the hydrogel surfaces after three days of incubation using trypsin. The dsDNA of fibroblasts retained within the hydrogel matrix was stained using fluorescent 4′,6-diamidino-2-phenylindole (DAPI) and enumerated revealing as strong correlation between the MPC content and the degree of fibroblast retention within the hydrogel. Hydrogel retention of RMS 13 cells was less than 1% for gels containing no PEGMA or MPC, ca. 10% for hydrogels containing 0.5 mol % PEGMA and no MPC, but ca. 80% for hydrogels containing both 0.5 mol % PEGMA and 10 mol % MPC.

To evaluate in vivo biocompatibility of our biomimetic hydrogel outer layer, cylindrical test hydrogel specimens (2 mm D and 2 mm T) (n=2 specimens per rat) were inserted into the medial trapezius muscle at the level of the mid quadriceps of a Sprague Dawley hemorrhage model (n=2 rats per specimen). Each of the hydrogel specimens (n=4) were found to elicit some degree of foreign body response.

It is important to note that none of the materials exhibited a “granulomatous” type of response that is characteristic of a very vigorous foreign body response. It is also important to note that the newly deposited connective tissue did not penetrate into the body of any of the hydrogels tested, but remained on the exterior. While only preliminary and not associated with the underlying electroconductive hydrogel membrane or the transducer, this evidence indicates the potential for extended implant biocompatibility of hydrogel compositions containing phosphorylcholine.

The inner electroconductive polymer layer (FIG. 4) produces a biorecognition membrane layer that localizes molecular biorecognition (enzymes) within an electroconductive poly(HEMA)-based polypyrrole hydrogel. Conductive electroactive polymers and hydrogels have been separately shown to support aspects of in vitro and in vivo biocompatibility such as an absence of cytotoxicity and excellent cell growth and proliferation. While electroconductive hydrogels have not been subjected to similar extensive investigation, they likewise promise similar, if not improved, in vitro and in vivo biocompatibility. After four days cells were found to have 100% viability in all cases. Cell proliferation however showed a marked difference between Au*|Gel-P(Py-co-PyBA) and all other reference samples. For both RMS13 and PC12 cell lines, there was a statistically significant (p=0.05) increase in cell proliferation on the ECH surfaces compared to the other modified gold surfaces. There was an 81% increase in RMS13 cell density at the end of the incubation period compared to a 12% increase in PC12. The RMS13 cells on the Au*|Gel-P(Py-co-PyBA) surface demonstrated mixed morphologies (spherical and spreading), while the PC12 cells were predominantly spherical with no evidence of neurite outgrowth. Of note was the fact that the cell densities associated with the Au*|PPy and Au*|Gel samples were both dissimilar to that of the Au*|Gel-P(Py-co-PyBA) and that the ECH presented a unique property that resulted in statistically greater cell grow and proliferation and that this correlated with the extent of electropolymerization of poly(pyrrole-co-4-(3-pyrrolyl)butyric acid) within the hydrogel.

Dynamic electrochemical and AC impedance characterization of the bioactive hydrogels and electroconductive hydrogels supported on microdisc electrode arrays has been reported. Multiple scan rate cyclic voltammetry (MSRCV) and electrical (2-electrode) and electrochemical (3-electrode) impedance spectroscopy were used to characterize the charge transfer characteristics of hydrogel layers on microdisc electrode arrays. MSRCV experiments were generally done using a PAR 283 Galvanostat/Potentiostat, and for EIS, was done when the PAR 283 was interfaced to a Solartron 1260 Frequency Response Analyzer (FRA). The conductivity of the electroconductive hydrogel varies with the extent of electropolymerization (8 μScm⁻¹ for 0.25 C./cm² and 76 μcm⁻¹ for 2.0 C./cm²) and the oxidation state; being more like the pristine hydrogel and capacitive at reducing potentials and being more polypyrrole-like and Ohmic at oxidizing potentials. The observations were reflected in the equivalent circuit parameters that describe both hydrogel coated and electroconductive hydrogel coated electrodes. Table 2 lists the impedance proprieties of various tissue types and compares these to bioactive and electroconductive hydrogels. Hydrogels closely match the impedance properties of the various tissue types and moreover may be engineered to perfectly match specific tissue types. In addition to modulating interfacial impedance, electroconductive hydrogels have also been demonstrated to contribute to interference suppression.

TABLE 2
Generalized impedance properties (real and imaginary) of various tissue
types compared to bioactive and electroconductive hydrogels.

| | Frequency of | Real | Imaginary |
|---|---|---|---|
| | Interrogation | Component | Component |
| Organs | (kHz) | (Ohms) | (Ohms) |
| Heart (Humans; without | 50 kHz | \|Z\| = 25.6 | |
| heart failure) | | | |
| Brain (Piglets; | 100 kHz | 50 | 15 |
| cerebral impedance) | | | |
| Kidney (Humans; | 50 kHz | 480-520 | 30-40 |
| undergoing hemodialysis) | | | |
| Liver (Rats; control group | 50 kHz | \|Z\| = 200 | |
| in liver stenosis study) | | | |
| Skeletal muscle (Bovine; | 50 kHz | 40 | 2.5 |
| healthy excised bovine tissue) | | | |
| Skin (Human; skin at 30° C.) | 50 kHz | 80 | 120 |
| Breast (Humans; healthy | 60 kHz | 130-180 | −10 to −30 |
| breast tissue) | | | |
| Lung (Humans; healthy group | 50 kHz | 514 | 52 |
| in lung cancer survey) | | | |
| Bioactive Hydrogels (Range | 50 kHz | 20-200 | 20-350 |
| of polymers: PVA, p(HEMA), | | | |
| Alginate) | | | |
| p(HEMA-co-PEGMA-co- | 50 kHz | 300-500 | −200.3 |
| HMMA) 3 mol % TEGDA | | | |
| Electroconductive | 50 kHz | 100 | −100 |
| PPy Hydrogel (200 mC/cm²) | | | |

Mechanical matching of the implanted device to the tissue bed is extremely important in supporting long term in-dwelling performance. It is believed that modulus mismatching across the device-tissue interface does, in response to micro-motions, exacerbate the foreign body response. Table 3 lists the dynamic mechanical properties (loss and storage moduli) of various tissue types and compares these to that of bioactive hydrogels. Hydrogels clearly have the potential to display a wide range of dynamic mechanical properties, but because of the potential to develop additional virtual cross links over time (gelation) these properties may be time-temperature dependent, may demonstrate freeze-thaw cycle rate dependency, and may change with environment (e.g. pH, divalent ions). Judicious manipulation of the cross link density, the molecular weight between crosslink, and the time-temperature processing history, provides a window into the design and control of the mechanical properties of the biorecognition membrane. A recent review emphasizing the mechanical properties of electroconductive hydrogels brings perspective to this emerging class of materials as bioactive interfaces.

TABLE 3
Generalized dynamic mechanical properties (shear storage
and loss moduli) of various tissue types compared to
bioactive and electroconductive hydrogels.

| | Shear Storage | Shear Loss |
|---|---|---|
| Organs | Modulus (kPa) | Modulus (kPa) |
| Heart | ~183 | 89.6-111 |
| | (Human; coronary | (Rabbit; cardiac |
| | arteries) | cells)[80] |
| Brain | 2.1-16.8 | 0.4-18.7 |
| Kidney | 1.4-6.8 | — |
| (pig kidney) | | |
| Liver | 0.75-3 | — |
| GI tract | 4.5-32 | 8-45 |
| | (Rat; small | (Human; |
| | intestine)^{[84]} | stomach) |
| Skeletal muscle | 22-115 | — |
| | (Rat; soleus muscle) | |
| Skin | ~5.0-7.0 | — |
| Breast | 130 | — |
| Lung | 0.25-1 | 0.1-1 |
| Bioactive Hydrogels | 0.4-1400 | 0.4-1300 |
| p(HEMA) | | |

The wireless dual potentiostat interfaces to the ECC MDEA 5037 dual electrochemical biotransducer with its glucose responsive electroconductive hydrogel region (GOx: Channel 1, Cell A) and (LOx: Channel 2, Cell B) for implantation into the trapezius muscle of Sprague Dawley rats.

The bioanalytical system of dual responsive electrochemical cell-on-a-chip MDEA 5037 electrodes, wireless transmitting dual potentiostat, receiver base station and software has been qualified for in vitro and in vivo use. The two channels of the dual potentiostat were tested with a pair of dummy (RC) cells that comprised a parallel arrangement of a 10 MΩ resistor and a 1.0 μF capacitor. Tested at 0.5 V the dummy cells were switched between channels and their responses averaged to yield Ch1=51.1+/−0.5 nA (n=6) and Ch2=54.4+/−0.2 nA (n=6) establishing an inherent 6.5% error between the channels. When similarly tested with 10 MΩ resistors, their responses averaged to yield Ch1=49.9+/−0.1 nA (n=6) and Ch2=49.6+/−0.1 nA (n=6) establishing an inherent 0.6% error between the channels.

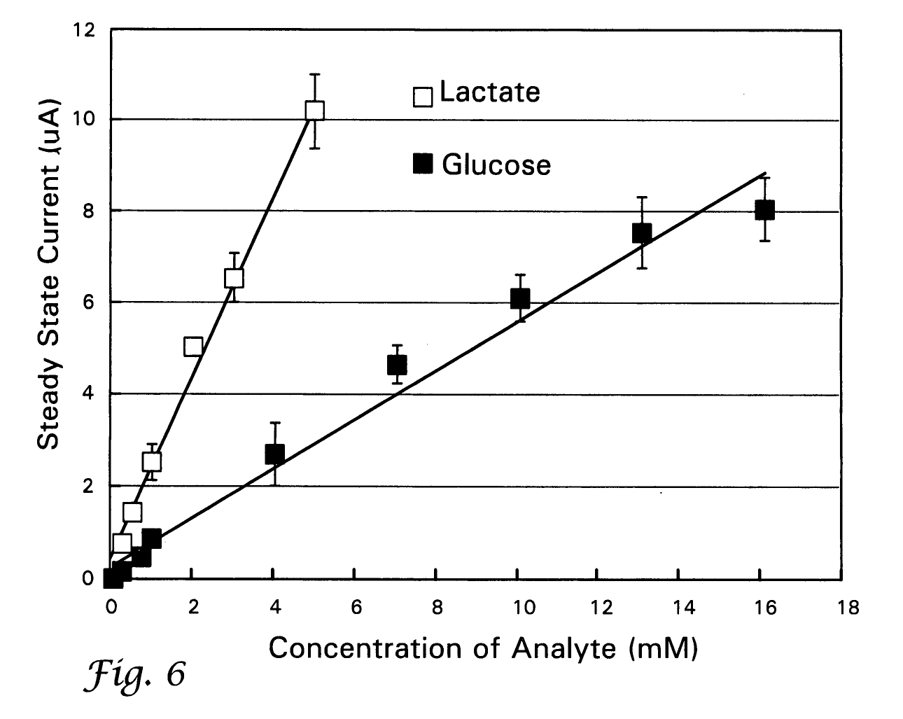

The bioactive electroconductive hydrogel biosensors were tested in vitro for their response to glucose and lactate. The exampled design uses a Type 1, non-mediated biosensor configuration. Parallel designs with free and covalently immobilized ferrocene monocarboxylic acid produced un-sustained amperometric responses believed to arise from instability of the ferrocenium ion. FIG. 6 shows the amperometric dose response of the glucose and lactate-specific biosensors to substrate challenges prepared in PBS (pH=7.4) at RT. Table 4 summarizes the key bioanalytical parameters determined for the un-optimized biosensors. The linear dynamic range for lactate measurements is inadequate to meet the needs of physiologic status monitoring during trauma induced hemorrhage. Basal lactate levels at typically 1.0 mmol/l and may be as high as 9.0 mmol/l in severely hemorrhaged patients. There is clearly need for improving the linear dynamic range for this analyte. The range of glucose concentrations for hypo- and hyperglycemic patients and diabetics (5-10 mmol/l) is adequately served by the liner dynamic range observed for the current biotransducers. However, among critically ill patients, particularly those subject to trauma induced hemorrhage, insulin resistance may result in glucose levels that range from (5-12 mmol/l) and are then candidates for conventional (blood glucose>12 mmol/l] or intensive insulin therapy to achieve euglycemia. Trauma patients may thus be adequately served by the observed linear dynamic range for glucose in the currently configured biotransducer.

TABLE 4
Bioanalytical Performance of the Bioactive Electroconductive
Hydrogels applied to the Measurement of Glucose and Lactate

| | |
|---|---|
| Glucose | Lactate |

| | | | | |
|---|---|---|---|---|
| Linear dynamic range | 0.1-13.0 | mM | 1.0-7.0 | mM |
| Sensitivity | 0.59 | μA/mM | 2.11 | μA/mM |
| Detection limit | 0.2 | mM | 0.4 | mM |
| Response time (t₉₅) | 50 | s | 35-40 | s |

Short term operational stability testing resulted in the glucose biotransducers producing 80% of their initial biotransducer response after 5 days of continuous storage at 37° C. and periodic testing of the biotransducer in 10 mM glucose.

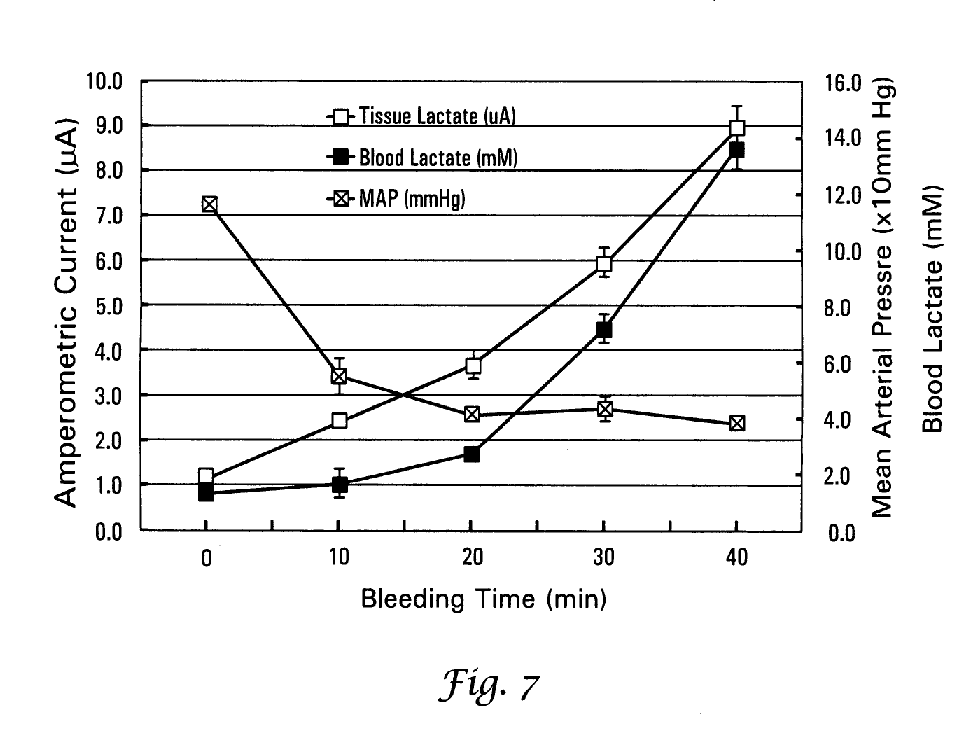

For preliminary in vivo bioanalytical studies, biotransducers were implanted into the trapezius muscle of a Sprague Dawley rat hemorrhage model under IACUC-approved protocols for small vertebrate animal surgery. Rats were first anesthetized (5% then 2-3% isoflurane, balance oxygen), prepped, and from a 2-5 cm long midline abdominal incision, a 3 to 5 Fr Silastic catheter was surgically inserted into the inferior vena cava and tunneled subcutaneously over the ribs toward the left side of the head. The catheter was terminated in a rodent sized vascular access port (VAP) placed in a subcutaneous pouch over the neck/scapular region. The VAP and catheter were filled with talurolidine citrate. To simulate hemorrhage and blood loss from trauma, blood was withdrawn from the rats at a rate of 2.5 ml/100 g/15 min from a femoral vein (to 40 ton) under isoflurane anesthesia. As the controlled hemorrhage occurred the onset of hemorrhagic shock and the changes in systemic and intramuscular glucose and lactate were observed. As expected, a total of 40-50% blood volume (ca. 6.5% of body weight) established a state of hemorrhagic shock. Systemic lactate from drawn blood was determined by electrochemical (amperometric) assay with enzyme membranes (ABL 705 Radiometer, Copenhagen, Denmark). Intramuscular lactate was determined from the implanted biotransducer. FIG. 7 shows the in vivo amperometric lactate response of the intramuscularly implanted biotransducer plotted alongside the systemic blood lactate values obtained using the ABL 705 blood gas and metabolite analyzer during hemorrhage (n=4). Intramuscular lactate levels are shown as amperometric current rather than as lactate concentration as this would imply equivalence between the in vitro calibration condition and the in vivo test condition, which has not been established. By trend inspection, intramuscular lactate levels are clearly discordant with systemic lactate levels and rise more rapidly during the early stages of hemorrhage.

Although the present invention has been described in connection with the preferred embodiments, it is to be understood that modifications and variations may be utilized without departing from the principles and scope of the invention, as those skilled in the art will readily understand. Accordingly, such modifications may be practiced within the scope of the following claims. Moreover, Applicant hereby discloses all subranges of all ranges disclosed herein. These subranges are also useful in carrying out the present invention.

What is claimed is:

1. An analyte measuring device for monitoring physiological status by measuring at least one analyte, comprising:
an insulating substrate;
a conductive material supported on the insulating substrate;
an insulating layer overlying the conductive material, the insulating layer formed in a pattern thereby passivating covered surface portions of the underlying conductive material and exposing predetermined surface portions of the conductive material, the exposed portions of the conductive material functioning as electrodes;
a polymeric biorecognition layer immediately adjacent to the insulating layer and the exposed surface portions of the conductive material, the polymeric biorecognition layer containing at least one molecular entity of biological or biomimetic origin that is specific to the at least one analyte being measured.

2. The device of claim 1 wherein the physiological status monitored comprises post-trauma status.

3. The device of claim 1 wherein the insulating substrate is selected from dielectric materials comprising polished borosilicate glass, oxidized silicon, alumina, polyamide, and silicone.

4. The device of claim 1 wherein the conductor is selected from the group comprising gold, platinum, palladium, iridium, indium tin oxide, polyaniline, polypyrrole and polythiophene.

5. The device of claim 1 wherein the insulating layer is selected from dielectric materials comprising silicon nitride, silicon oxide, spin-on-glass, and polyimide.

6. The device of claim 1 wherein the polymeric recognition layer comprises a hydrated hydrogel.

7. The device of claim 1 wherein the molecular entity of biological or biomimetic origin comprises a member selected from the group comprising DNA, RNA, enzymes, antibodies, antibody fragments, and antibody-linked enzymes, and wherein the molecular entity of biological or biomimetic origin is covalently attached to the polymer of the polymeric biorecognition layer.

8. The device of claim 7 wherein the molecular entity of biological or biomimetic origin comprises an enzyme, the enzyme being chemically modified by the covalent attachment of methacryloyl groups.

9. The device of claim 8 wherein the enzyme is selected from lactate oxidase, glucose oxidase, and laccase.

10. A biocompatible biosensor and transmitter device for temporary implantation prior to, during and following trauma-induced hemorrhaging, the device detecting the presence and level of at least one analyte and transmitting detected data to a second, external data receiving device.

11. The biocompatible biosensor and transmitter device set forth in claim 10 wherein the at least one analyte comprises lactate.

12. The biocompatible biosensor and transmitter device set forth in claim 11 wherein the at least one analyte comprises glucose.

13. The biocompatible biosensor and transmitter device set forth in claim 11 wherein the at least one analyte comprises oxygen.

14. The biocompatible biosensor and transmitter device set forth in claim 11 wherein the at least one analyte comprises H⁺ cations for detecting pH.

15. A method for managing post-trauma patient outcomes comprising:
providing a biocompatible biosensor and transmitter device and a data receiving means, the biocompatible biosensor and transmitter device capable of detecting the presence and level of at least one analyte and transmitting detected data to the data receiving means;
temporarily implanting the biocompatible biosensor and transmitter device into a muscle of a hemorrhaging trauma victim; and
employing the biosensor of the biocompatible device to collect data regarding the presence or the amount of the at least one analyte and employing the transmitter of the biocompatible device to transmit data to the data receiving means.

16. The method set forth in claim 15 wherein the data receiving means is programmed for processing and presenting the received data.
